(19) United States
(12) Patent Application Publication
(10) Pub. No.: US 2025/0058314 A1
(43) Pub. Date: Feb. 20, 2025

(54) APPARATUS COMPRISING CARTRIDGE WITH ABSORBING ELEMENT

(71) Applicant: Radiometer Medical ApS, Brønshøj (DK)

(72) Inventors: Michael Frank Petersen, Brønshøj (DK); Linda Soland Engbæk, Brønshøj (DK); Søren Damgård Ravn, Brønshøj (DK)

(21) Appl. No.: 18/709,163

(22) PCT Filed: Nov. 11, 2022
(86) PCT No.: PCT/EP2022/081669

§ 371 Date: May 10, 2024

(30) Foreign Application Priority Data
Nov. 11, 2021 (EP) 21207822.4

(51) Int. Cl. B01L 3/00

(52) U.S. Cl. CPC B01L 3/502715; B01L 3/5023; B01L 2200/026; B01L 2200/04; B01L 2200/16; B01L 2300/069; B01L 2300/123

(57) ABSTRACT

There is presented an apparatus for analyzing one or more fluids and comprising an analysis unit for analyzing one or more fluids, such as human blood, a liquid inlet port, and a liquid outlet port, a cartridge comprising a pouch comprising a liquid, such as an aqueous liquid, an absorbing element, wherein the absorbing element is capable of absorbing water, and wherein the absorbing element is arranged exterior to the pouch and fluidically connected to an exterior surface of the pouch, a liquid outlet port being fluidically connected to the pouch, and a liquid inlet port being fluidically connected to the element, wherein the liquid inlet port of the analysis unit is fluidically connected with the liquid outlet port of the cartridge, and the liquid outlet port of the analysis unit is fluidically connected with the liquid inlet port of the cartridge.

### FIELD OF THE INVENTION

The present invention relates to an apparatus for analyzing one or more fluids, more particularly an apparatus for analyzing one or more fluids and comprising a cartridge, and furthermore relates to a corresponding cartridge, method and use.

### BACKGROUND OF THE INVENTION

A fluid analysis apparatus can be used in testing and measuring, e.g., gas and/or electrolyte levels in fluids. Such apparatus may, for example, be used in medical and environmental applications to measure the gas and/or electrolyte levels in blood, urine, water, and other fluids.

It may be relevant to rinse and/or calibrate such fluid analysis apparatus, optionally repeatedly or even frequently, when they are being used to test fluid samples. For example, it may be preferable to rinse and/or calibrate, and optionally re-calibrate, a blood analyzing apparatus after each blood sample is tested for gas and/or electrolyte levels. As a result, it may be relevant to provide some means of applying a rinsing and/or a calibration (or reference) solution to the fluid analysis apparatus. Additionally, it may be relevant or required to handle, such as receive and store, waste liquid, which may potentially be biohazardous.

Therefore, there is a need for an improved fluid analysis apparatus, and corresponding cartridge, method and use, for facilitating providing some means of applying a solution, such as a rinsing and/or a calibration (or reference) solution, to the fluid analysis apparatus and/or receiving a (waste) liquid.

### SUMMARY OF THE INVENTION

An object of the present invention is to provide an improved fluid analysis apparatus, and corresponding cartridge, method and use, for facilitating providing some means of applying a solution, such as a rinsing and/or a calibration (or reference) solution, to the fluid analysis apparatus and/or receiving a (waste) liquid. Another object of the present invention may be to provide an alternative to the prior art.

According to a first aspect, the invention provides an apparatus for analyzing one or more fluids, the apparatus comprising:
An analysis unit for analyzing one or more fluids, such as a body fluid, such as comprising or being blood, such as human blood, the analysis unit comprising:
   i. A liquid inlet port, and
   ii. A liquid outlet port, and
a cartridge comprising:
   i. A pouch, such as one or more pouches, such as two or more pouches, such as three or more pouches, such as 4 or more pouches, such as 5 or more pouches, each comprising a liquid, such as an aqueous liquid,
   ii. An absorbing element, wherein the absorbing element is capable of absorbing water, such as capable of absorbing liquid water and/or liquid blood, and wherein the absorbing element is arranged exterior to the pouch and fluidically connected to an exterior surface of the pouch,
   iii. A liquid outlet port being fluidically connectable to the pouch and/or fluidically connected to the pouch, and
   iv. A liquid inlet port being fluidically connectable to the absorbing element and/or fluidically connected to the absorbing element,
      wherein
the liquid inlet port of the analysis unit is fluidically connected with the liquid outlet port of the cartridge, and
the liquid outlet port of the analysis unit is fluidically connected with the liquid inlet port of the cartridge.

A possible advantage of the present invention is that by having the absorbing element being capable of absorbing water and arranged exterior to the pouch and fluidically connected to an exterior surface of the pouch, the absorbing element can absorb liquid(s) leaked from the pouch, e.g., avoiding that said liquid is leaked from the cartridge. According to an embodiment, the absorbing element is arranged external to each and all of the one or more pouches of the cartridge. It is to be understood, that such embodiments do not exclude that an additional absorbing element may be present in at least one of the one or more pouches of the cartridge and/or that an additional absorbing element may be present in at least one of one or more (additional) pouches of the cartridge.

Another possible advantage is that the absorbing element can (also) absorb waste liquid from the analysis unit, such as waste liquid introduced into the cartridge via the liquid inlet port of the cartridge. This may in particular be relevant in the case of a biohazardous (waste) liquid, such as a liquid being or comprising animal or human blood, such as mammalian and/or human blood, such as human blood, which must be contained in the cartridge to avoid exposure to personnel handling the analysis unit and/or the cartridge.

Another possible advantage is that the absorbing element might dispense with a need for a (separate) pouch for, e.g., waste liquid, which may in turn be advantageous for saving costs and/or space.

By an ‘apparatus for analyzing one or more fluids’ may be understood any apparatus enabling analyzing one or more fluids (such as determining a parameter, such as a concentration, or a value indicative of a concentration, of one or more analytes, such as ions, in the one or more fluids), such as a spectrometric analysis setup for aqueous liquids or an electroanalytical measurement setup, wherein an ‘electroanalytical method’ is a method for chemical analysis, such as a method enabling determining a concentration, or a parameter indicative of a concentration, of an analyte, such as a reference ion and/or an analyte ion, in a liquid sample, by an electrolytic method, such as potentiometry, amperometry, conductometry, electrogravimetry, voltammetry (and polarography), and coulometry.

By ‘analyzing one or more fluids’ may be understood determining partial pressure of a gas, such as the partial pressure of oxygen (pO₂) or carbon dioxide (pCO₂), the content of O₂ and/or CO₂ in a gas, a concentration or partial pressure of a parameter, such as any one or more of pH, pO₂, pCO₂, K⁺, Na⁺, Ca²⁺, Cl⁻, Glucose, Lactate, Urea, Creatinine, tHb (total hemoglobin), O₂Hb (oxygen-carrying hemoglobin), COHb (carboxyhemoglobin), MetHb (Methemoglobinemia) and bilirubin.

By ‘analysis unit’ may be understood the part of the apparatus excluding the cartridge, such as wherein the analysis unit comprises one or more or all of a fluidic system, such as a microfluidic system, a user interface and/or one or more ports for input and output of information and controlling signals, a measurement cell, such as a measurement cell for an electroanalytical method.

The analysis unit may be a stop flow system, such as a system wherein a flow is stopped during updating of sensors (which may be advantageous for, e.g., blood gas measurements). Typically, it is advantageous or even necessary to use a stop flow system due to sensors with slow response or due to electrical interaction from pumps, etc.

The analysis unit may be or comprise an electroanalytical measurement setup, such as an ion selective electrode sensor, such as an ion selective electrode membrane sensor.

‘Electroanalytical measurement setup’ is understood as is common in the art, such as a setup for an electroanalytical method, such as wherein an ‘electroanalytical method’ is a method for chemical analysis, such as a method enabling determining a parameter indicative of a concentration of an reference ion and/or an analyte ion in a liquid sample, by an electrolytic method, where ‘electrolytic method’ is understood as is common in the art, such as a method for producing chemical changes by application of an electrical potential and/or passage of an electric current through an electrolyte. More particularly, it is understood that an electroanalytical method determines the parameter indicative of concentration of the analyte ion and/or the reference ion by measuring an electrical potential (volts) and/or current (amperes) in an electrochemical cell containing the analyte ion and/or reference ion. An electroanalytical method uses electrically conductive probes, called electrodes, to make electrical contact with the analyte solution. The electrodes are used in conjunction with electric or electronic devices to which they are attached to measure the parameter of the solution. The measured parameter is related to the identity of the analyte ion or reference ion and/or to the quantity of the analyte ion or reference ion in the solution. Electroanalytical methods include potentiometry, amperometry, conductometry, electrogravimetry, voltammetry (and polarography), and coulometry.

By ‘liquid inlet/outlet port’ may be understood a port for allowing liquid to flow, respectively, into and out of out of an entity, such as the analysis unit. It is generally understood that liquid inlet/outlet port can be separate (such as fluidically disconnected, e.g., due to being separate openings) or shared (such as being fluidically connected, e.g., due to sharing the same opening).

‘Cartridge’ is understood as is common in the art, such as a case or container that holds a liquid and/or is capable of receiving a liquid. It may furthermore be understood that a cartridge can form part of a larger system, such as an apparatus, wherein the cartridge may be exchanged with another (similar) cartridge, which may be advantageous for changing the liquids (for being supplied to the apparatus) and/or for regaining a capability of receiving liquid (from the apparatus), which may be beneficial for mitigating a problem of handling liquid, which might otherwise be difficult, troublesome, awkward, or hazardous to handle. In the present context, the cartridge is understood to have a size sufficiently large for enabling offering the relevant functions, yet sufficiently small to be practical and portable. The cartridge may be understood to have a size along its largest outer dimension of 1 meter or less, such as 50 cm or less, or such as 25 cm or less, and/or a minimum size along its largest dimension of 5 cm or more, such as 10 cm or more. A mass of the cartridge may be 10 kg or less, such as 5 kg or less, or such as 1 kg or less, and/or a mass of the cartridge may be 100 g or more, such as 500 g or more, such as 1 kg or more, such as 5 kg or more. A material of boundary walls of the cartridge may comprise or consist of polymer, such as predominantly (by mass) comprise polymer. A material of boundary walls of the cartridge may comprise or consist of cardboard, such as cardboard with an internal lining material of polymer, such as predominantly (by mass) comprise cardboard.

According to an embodiment, there is presented a cartridge, wherein the cartridge is having a size along its largest outer dimension of 25 cm or less. An advantage of this may be that the cartridge is sufficiently small to be practical and portable.

According to an embodiment, there is presented a cartridge, wherein the cartridge is having a size along its largest outer dimension of 5 cm or more. An advantage of this may be that the cartridge is sufficiently large for enabling offering the relevant functions.

According to an embodiment, there is presented a cartridge, wherein the cartridge is having a size along its largest outer dimension of 5 cm or more and 25 cm or less. An advantage of this may be that the cartridge is sufficiently large for enabling offering the relevant functions, yet sufficiently small to be practical and portable.

By ‘pouch’ may be understood a bag for comprising a liquid, such as a bag as described in any one of EP3181354 A1, WO9743988 A1, WO08131768 A1 or WO18184902 A1, which are each incorporated by reference in entirety. While reference is made to “a pouch” and “the pouch”, multiple pouches as well as a single pouch are each conceivable and within the scope of the present invention, i.e., ‘a pouch’ and ‘the ‘pouch’ can be used interchangeably with ‘at least one pouch’ or ‘one or more pouches’, respectively, ‘the at least one pouch’ or ‘the one or more pouches’. In embodiments with multiple pouches, ‘a pouch’ and ‘the ‘pouch’ can be replaced with ‘multiple pouches’ or ‘a plurality of pouches’, respectively, ‘the multiple pouches’ or ‘the plurality of pouches’.

The one or more pouches each comprises a liquid, such as an aqueous liquid, which may be applicable for rinsing and/or calibration of the analysis unit. The liquid may be a solution. By ‘solution’ is understood a liquid comprising a dissolved substance, such as a gas and/or a salt.

An advantage of having multiple pouches may be that it enables multiple functions (such as rinsing and calibration), and/or a multi-point (such as a two-point) calibration.

Some parameters can be calibrated with only one calibration liquid, but in general, a minimum of two different liquids are necessary for a sensitivity calibration. Thus, in case a separate rinsing liquid is required and/or a multi-liquid calibration is required, multiple pouches are advantageous for enabling handling and/or providing the multiple liquids.

Five or more pouches may be relevant, e.g., for providing a quality control liquid, three liquids for calibration and a rinsing liquid.

It may be understood that in embodiments there is a plurality of pouches, each having a unique liquid with respect to the other pouches within said plurality of pouches.

By ‘absorbing element’ may be understood an absorbing element being capable of absorbing water, such as being capable of absorbing at least a mass of water corresponding to its own mass, such as 2 or more times its own mass, such as 5 times or more its own mass, such as 10 times or more its own mass, such as 50 times or more its own mass, such as 100 times or more its own mass, such as 500 times or more its own mass, such as 1000 times or more its own mass.

By ‘arranged exterior to the pouch’ may for example be understood that the absorbing element can absorb liquid subsequent to said liquid leaking outside of the pouch.

By ‘fluidically connected to an exterior surface of the pouch’ is for example to be understood that a fluid or a liquid can travel from an exterior surface of the pouch (such as from a point of leakage) to the absorbing element, e.g., (in case the pouch and absorbing element are spatially separated) under the influence of gravity and/or capillary forces.

By ‘liquid outlet port (of the cartridge) being fluidically connected to the pouch’ is understood means for fluidically connecting, such as fluidically connecting, the pouch (such as the exterior or interior of the pouch) with an entity (such as an analysis unit) external to the cartridge, such as an opening. The liquid outlet port of the cartridge may comprise a valve or simply be an opening, such as an opening in a casing of the cartridge separating the pouch from the exterior of the cartridge. The liquid outlet port of the cartridge may be arranged so that liquid originating from the pouch can exit the cartridge via the liquid outlet port of the cartridge, optionally led within a tube or a tubing. The cartridge may furthermore comprise a fluid-tight connection, such as a tube or a tubing, for fluidically connecting the pouch with the liquid outlet port of the cartridge (such as wherein liquid in the pouch can arrive at the liquid outlet port of the cartridge in a fluid-tight manner, such as without liquid in the pouch leaking to the interior of the cartridge and/or so that liquid for entry into the analysis unit is not mixed with liquid exiting the analysis unit).

By ‘liquid inlet port (of the cartridge) being fluidically connected to the absorbing element’ is understood means for fluidically connecting, such as fluidically connecting, the absorbing element with an entity (such as an analysis unit) external to the cartridge. The liquid inlet port of the cartridge may comprise a valve or simply be an opening, such as an opening in a casing of the cartridge separating the absorbing element from the exterior of the cartridge. The liquid inlet port of the cartridge may be arranged so that liquid can enter the cartridge via the liquid inlet port of the cartridge, optionally led within a tube or a tubing. The cartridge may furthermore comprise a fluid-tight connection, such as a tube or a tubing, for fluidically connecting the absorbing element with the liquid inlet port of the cartridge (such as wherein liquid in the pouch can arrive at the absorbing element from the liquid inlet port of the cartridge in a fluid-tight manner, such as without liquid from the liquid inlet port of the cartridge going elsewhere in the interior of the cartridge prior to arriving at the absorbing element).

By ‘the liquid inlet port of the analysis unit is fluidically connected with the liquid outlet port of the cartridge’, and ‘the liquid outlet port of the analysis unit is fluidically connected with the liquid inlet port of the cartridge’ is understood, respectively, that the respective ports are fluidically, such as fluidically and operatively (such as fluid-tightly), connected, such as enabling fluid flow from the pouch to the analysis unit and from the analysis unit and to the absorbing element. It may in particular be understood that the apparatus may be arranged to enable (such as ensure) fluid-tight flow from the pouch to (such as via the liquid outlet port of the cartridge) the analysis unit (such as the liquid inlet port of the analysis unit, and optionally through the analysis unit to the liquid outlet port of the analysis unit) and/or arranged to enable (such as ensure) fluid-tight flow from the analysis unit (such as the outlet port of the analysis unit) to the liquid inlet port of the cartridge.

According to an embodiment, there is presented an apparatus wherein the one or more fluids, such as the one or more fluids which the apparatus is for analyzing, include an aqueous liquid with one or more inorganic constituents, such as a bio-mimicking fluid or a biological fluid, such as blood, such as animal blood or human blood, such as mammalian and/or human blood, such as human blood. Examples of aqueous liquids with one or more inorganic constituents include cerebrospinal liquid, urine, cell culture liquid, pharmaceutical liquids, beer production liquid or any aqueous liquid containing O₂ and/or CO₂. It may furthermore be understood that the liquid may be at least in part, such as in part or in entirety: a whole blood sample, such as an animal (such as mammalian) or human whole blood sample, or a sample derived from a whole blood sample, such as serum or plasma, such as derived from a whole human blood sample, such as being a diluted whole blood sample and/or being a fraction of a whole human blood sample.

According to an embodiment, there is presented an apparatus, wherein the one or more fluids comprise (such as wherein a whole human blood sample is provided outside or at the apparatus and is mixed outside or inside the apparatus with one or more other fluids before the resulting mixture is being analyzed by the apparatus) or is human blood, such as whole human blood or a sample derived from a whole human blood sample (such a part of, e.g., a plasma or a serum of, a whole human blood sample).

According to an embodiment, there is presented an apparatus, wherein the one or more fluids include blood, such as human blood, and wherein the apparatus is suitable for determining one or more blood parameters, such as one or more of pH, pO₂, pCO₂, K⁺, Na⁺, Ca²⁺, Cl⁻, Glucose, Lactate, Urea, Creatinine, tHb (total hemoglobin), O₂Hb (oxygen-carrying hemoglobin), COHb (carboxyhemoglobin), MetHb (Methemoglobinemia) and Bilirubin

According to an embodiment, there is presented an apparatus, wherein the analysis unit is suitable for determining one or more blood gas parameters, such as one or more of pH, pO₂, pCO₂, such as wherein the analysis unit is a stop flow system.

According to an embodiment, there is presented an apparatus, wherein the analysis unit comprises an ion selective electrode membrane sensor, such as an ion selective electrode membrane sensor adapted for sensing one or more blood gas parameters. ‘Ion-selective electrode’ (ISE) is understood as is common in the art. The ISE may be selective for a single ion or a group of ions. More particularly, an ISE is an electrochemical sensor or electrode that allow potentiometric determination of the activity of a certain ion in the presence of other ions. An ISE may comprise an ion-selective membrane allowing passage of only (taking into account possibly some cross-selectivity as described in the following) the selected ion to a conducting, internal electrode. ISEs might encompass electrodes which are somewhat (cross-) selective to one or more interfering ions. However, an ISE may be understood to be more selective to an ion or a group of ions compared to another ion or group of ions (such as a selectivity coefficient of the interfering ions being less than 1.0, such as less than 0.9, such as less than 0.5, such as less than 0.1, such as less than 0.07, such as less than 0.05, such as less than 0.03, such as less than 0.02, such as less than 0.01).

The ‘membrane’ is fully or partially solid but may also contain a plasticiser (such as wherein the remainder is partially or fully a liquid), such as comprises at least 20 volume/volume percent (v/v %) solid matter, such as comprises at least 40 v/v % solid matter, such as comprises at least 60 v/v % solid matter, such as comprises at least 80 v/v % solid matter such as comprises at least 90 v/v % solid matter, such as comprises at least 95 v/v % solid matter, such as comprises at least 99 v/v % solid matter, such as is fully solid.

According to an embodiment, there is presented an apparatus, wherein the cartridge is detachable from the analysis unit, such as reversibly connected to the analysis unit. By ‘detachable’ may be understood detachable in a non-destructive manner, such a detachable and optionally reversibly connectable without necessarily destroying or damaging any part of the analysis unit and/or the cartridge. In embodiments, the cartridge may be detachable and optionally reversibly connectable by hand (such as enabling detachment without use of any tools) and/or detachable by a person without using tools. An advantage of having a detachable cartridge may be that it enables removing the cartridge, which may in turn enable providing another cartridge to the analysis unit. A possible advantage of the cartridge being detachable and optionally reversibly connectable by a person without using any tools may be, that it enables relatively easily detaching and optionally reversibly connecting the cartridge. By ‘without using tools’ may be understood, that the detachment and optional reversibly connection can be carried out with the bare hands, such as with the bare hands of an average adult person. In a further embodiment the analysis unit and cartridge can be fixed together by a transition fit, such as a reversible friction fit. By a ‘transition fit’ is understood a fit where the parts to be held together are held securely, yet not so securely that it cannot be disassembled, such as disassembled without tools, such as disassembled by the hands of a human, such as an average adult person. In a further embodiment, the analysis unit and cartridge can be fixed together by a mechanical locking member, such as one or more or all of: a pin, such as a split pin or a spring pin, a click-lock, such as a lock wherein a spring loaded engagement member positioned on one part engages with a cavity or edge on another part upon assembly so that the spring force has to be overcome before disassembly, a detent ball, a hand-operable screw, such as a tommy screw, or a wing screw.

According to a second aspect, there is provided a cartridge comprising:
A pouch, such as one or more pouches, such as two or more pouches, such as three or more pouches, such as 4 or more pouches, such as 5 or more pouches, each comprising a liquid, such as an aqueous liquid,
An absorbing element being capable of absorbing water, such as capable of absorbing liquid water and/or liquid blood, and arranged exterior to the pouch and fluidically connected to an exterior surface of the pouch,
A liquid outlet port (of the cartridge) being fluidically connected to the pouch, (and)
A liquid inlet port (of the cartridge) being fluidically connected to the absorbing element.

According to an embodiment, there is presented a cartridge, wherein the absorbing element is arranged external to each pouch (such as the one or more pouches) of the cartridge, such as external to each and all pouches (such as the one or more pouches) of the cartridge. It is to be understood, that such embodiment does not exclude that an additional absorbing element may be present in at least one of the pouches of the cartridge and/or that an additional absorbing element may be present in at least one of one or more (additional) pouches of the cartridge. An advantage may be that a pouch for encapsulating the absorbing element is dispensed with and/or that the absorbing element is available for absorbing leaked liquid from each pouch of the cartridge, such as from (the) one or more (other) pouches.

According to an embodiment, there is presented a cartridge, wherein the absorbing element is not being encapsulated by a pouch, such as not being encapsulated by the pouch, such as not being encapsulated by the one or more pouches. An advantage may be that a pouch for encapsulating the absorbing element is dispensed with and/or that the absorbing element is available for absorbing leaked liquid from one or more other pouches, such as from the one or more (other) pouches.

According to an embodiment, there is presented a cartridge comprising two or more pouches, each comprising a liquid, such as an aqueous liquid, and wherein the cartridge is further comprising a liquid outlet port being fluidically connectable to each pouch and/or fluidically connected to each pouch. According to an embodiment, there is presented a cartridge comprising four or more pouches, each comprising a liquid, such as an aqueous liquid, and wherein the cartridge is further comprising a liquid outlet port being fluidically connectable to each pouch and/or fluidically connected to each pouch. According to an embodiment, there is presented a cartridge comprising five or more pouches, each comprising a liquid, such as an aqueous liquid, and wherein the cartridge is further comprising a liquid outlet port being fluidically connectable to each pouch and/or fluidically connected to each pouch. An advantage of each of these embodiments may be that it enables that liquids from two, three, four, five or more pouches may be supplied from the cartridge, which may in turn enable that different liquids may be supplied from the cartridge and/or that liquids may be supplied from pouches being opened at separate times, which may enable supplying (fresh) liquid for a longer time. It may be understood that liquids may be supplied from each pouch in a liquid-tight manner, so fluids from separate pouches is not mixed (such as the liquid outlet port comprising a plurality of sub-liquid outlet ports, with each of the pouches being arranged for being fluidically connected with a dedicated sub-liquid outlet port).

According to an embodiment, there is presented a cartridge, wherein the cartridge comprises an enclosure, wherein the enclosure is at least enclosing:
The pouch, such as the one or more pouches,
The absorbing element,
and wherein optionally:
The liquid outlet port is accessible from outside of the enclosure,
The liquid inlet port is accessible from outside of the enclosure.

An advantage of such enclosure may be that it enables dispensing with a trade-off in terms of material properties of an outer shielding (such as the enclosure) of the contents of the cartridge and an inner shielding (such as the pouch) of the liquid(s). For example, a first element of a first material can be optimized for the enclosure (e.g., relatively rigid, self-supporting polymeric plates for ensuring a fixed shape of the cartridge) and a second element of a second material can be optimized for the pouch (e.g., relatively flexible, non-self-supporting fluid- and gas-tight laminates, comprising a metallic foil). Another possible advantage may be that the enclosure enables forming a unit (as observed from outside of the enclosure) comprising (internally) a plurality of parts (including pouch and absorbing element), which may optionally facilitate and/or simplify handling.

By ‘enclosure’ is understood something that enclosures, such as being or comprising a case, a casing, a box and/or a container. The enclosure may comprise planar surface, such as a polyhedron, such as a trapezoidal prism, such as a cuboid.

According to an embodiment, there is presented a cartridge, wherein the enclosure is more rigid than the pouch. A possible advantage of this may be that the larger rigidity of the enclosure simplifies and/or facilitates handling, e.g., due to the outer shape being more well-defined and/or due to insertion (and optionally) removal from an analysis apparatus becoming simpler, easier and or more secure (e.g., due to the shape of the cartridge being more predictable, which may in turn enable making a slot in the analysis unit corresponding to the shape of the cartridge). By ‘more rigid’ may in this context be understood in the context of rigidity of the shape of the pouch, respectively, the enclosure. By ‘more rigid’ may be understood that a compressive and/or flexural modulus of the enclosure is higher, such as at least 10% higher, such as at least 50% higher, such as at least 100% higher, such as at least 1000% higher, than a compressive and/or flexural modulus of the pouch. For example, less force is required to push two distal ends of the pouch towards each other than two ends of the enclosure and/or less force is required to bend a (planar portion or a wall) of the pouch than of the enclosure.

According to an embodiment, there is presented a cartridge, wherein the enclosure is self-supporting, such as self-supporting in one or more or all orientations. A possible advantage is that the shape of the cartridge maintains constant, such as even if the orientation is changed. By ‘self-supporting’ is understood that gravitational forces at the surface of the earth are insufficient for substantially deforming the shape (of the cartridge), such as for deforming the shape (of the cartridge).

According to an embodiment, there is presented a cartridge, wherein the liquid outlet port, such as the liquid outlet port of the cartridge, is fluidically connected with the liquid inlet port, such as the liquid inlet port of the cartridge, such as wherein a single liquid port can double function as both liquid inlet port and liquid outlet port. An advantage may be that a single liquid port can double function as both liquid inlet port and liquid outlet port.

According to an embodiment, there is presented a cartridge, wherein the liquid outlet port, such as the liquid outlet port of the cartridge, is fluidically separated with respect to the liquid inlet port, such as the liquid inlet port of the cartridge. An advantage may be that a liquid in either port does not mix with, such as become contaminated with and/or diluted by, a liquid in the other port.

According to an embodiment, there is presented a cartridge, wherein the pouch and absorbing element are arranged so that in case liquid initially comprised within the pouch is leaked to the exterior of the pouch, said liquid initially comprised within the pouch is able to be absorbed by the absorbing element subsequent to leaking to the exterior of the pouch, such as at least in one orientation of the cartridge. A possible advantage is that the absorbing element can absorb liquid leaked from the pouch, which may be beneficial for fixating the liquid within the cartridge, which may in turn be beneficial for avoiding liquid leaking out of the cartridge. It may be understood that liquid leaked from the pouch can move by itself, such as under the influence of gravity (in at least one orientation of the cartridge, such as a standard orientation during normal use of an apparatus, such as an apparatus comprising an analysis unit and the cartridge, such as with the cartridge installed in the apparatus) and/or capillary forces without reorienting the cartridge, to the absorbing element. This may be advantageous for dispensing with a need for moving the liquids by other means, such as by pumping or having to reorient the cartridge. It may be understood, that in a specific embodiment, the absorbing element could be arranged so that liquid leaked from the pouch is absorbed regardless of the orientation of the cartridge (e.g., by having the absorbing element encapsulate the pouch). However, it may be more generally understood, that liquid leaked from the pouch is able to be absorbed by the absorbing element at least in one orientation of the cartridge, such as an orientation of the cartridge during normal use, e.g., with the absorbing element being arranged so that gravity and/or capillary forces can drive leaked liquid from the pouch to the absorbing element.

According to an embodiment, there is presented a cartridge, wherein the pouch, such as the one or more pouches, and the absorbing element are arranged so that in case liquid initially comprised within the pouch, such as the one or more pouches, is leaked to the exterior of the pouch, such as the one or more pouches, through an opening in the pouch, such as the one or more pouches, anywhere in an area covering at least 25%, such as at least 50%, such as at least 75%, such as at least 90%, such as 100%, of the surface of the pouch, such as the one or more pouches, said liquid initially comprised within the pouch is able to be absorbed by the absorbing element subsequent to leaking to the exterior of the pouch, such as the one or more pouches, such as at least in one orientation of the cartridge. An advantage may be that liquid from leakages from a relatively large area of the pouch, such as the one or more pouches, can be absorbed by the absorbing element.

According to an embodiment, there is presented a cartridge, wherein the pouch comprises a flexible part, such as a flexible laminate, such as a flexible multi-layered laminate, arranged so that a volume of the pouch diminishes upon removal of liquid from the pouch, such as emptying of the liquid initially in the pouch. By ‘volume of the pouch diminishes’ may be understood that a total volume of the pouch, such as the sum of the internal volume (such as the space inside the pouch originally being occupied by liquid, and subsequently evacuated and/or reduced in size) and the volume of the pouch material, diminishes by an amount substantially corresponding to the volume of liquid emptied out of the pouch or by an amount corresponding to at least 10%, such as at least 25%, such as at least 50%, such as at least 75%, such as at least 90%, of the liquid emptied out of the pouch. A possible advantage is that the diminished volume may leave space outside the pouch for other purposes, such as for an expanding absorbing element (upon absorption of waste liquid). By ‘flexible (part)’ may in this context be understood that it is sufficiently flexible that a total volume of the pouch can be reduced (or compressed) by at least 10%, such as at least 25%, such as at least 50%, such as at least 75%, without the walls of the pouch breaking and/or leaking. In embodiments, an external pressure outside of the pouch, such as an external atmospheric pressure (such as 1-2 atmosphere or 101325-202650 Pa) outside of the pouch, can compress the pouch, such as reduce a volume of the pouch by at least 10%, such as at least 25%, such as at least 50%, such as at least 75%, upon removal of the liquid in the pouch. The pouch may be fluid-tight so that upon removing liquid from the pouch, then fluid (initially external to the pouch) will not partially or fully enter the pouch (so as to replace the removed liquid).

According to an embodiment, there is presented a cartridge, wherein the pouch and the absorbing element are arranged so that a volume initially occupied by the pouch can subsequently become occupied by the absorbing element, such as arranged so that a volume initially occupied by the pouch will subsequently become occupied by the absorbing element, via
removal of liquid from the pouch, such as emptying of the liquid initially placed in the pouch, and
absorption of liquid by the absorbing element.

For example, upon removing liquid from the pouch, e.g., by supplying (rinsing or calibrating) liquid to an associated analysis unit, a volume of the pouch diminishes and simultaneously and/or subsequently, the absorbing element increases in size, e.g., due to swelling, due to absorption of liquid, such as waste liquid from an associated analysis unit, and wherein a volume initially occupied by the pouch can-upon the volume of the pouch being diminished—subsequently become occupied by the absorbing element. An advantage of this may be, that the cartridge can be kept relatively small due to the double functioning (part of the) internal volume (i.e., firstly being occupied by the pouch and subsequently being occupied by the absorbing element). The skilled person will be able to determine if a volume initially occupied by the pouch will subsequently become occupied by the absorbing element, e.g., e.g., by knowledge about increases/decreases in volume and/or by visual observation and/or by measuring, e.g., with rulers or laser equipment if a volume initially occupied by the pouch will subsequently become occupied by the absorbing element.

According to an embodiment, there is presented a cartridge, wherein
the pouch is arranged (such as due to the pouch comprising a flexible part arranged so that a volume of the pouch diminishes upon removal of liquid from the pouch) so that upon removal of liquid from the pouch, such as removal of an amount of liquid from the pouch, such as emptying of the liquid initially placed in the pouch, the volume occupied by the pouch will change from a first pouch volume to a second pouch volume, such as wherein the second pouch volume is smaller than the first pouch volume, and
the absorbing element is arranged (such as due to the absorbing element comprising a material swelling upon liquid absorption, such as any of cellulose acetate, bentonite, a hydrocolloid and/or sodium polyacrylate) so that upon absorption of liquid by the absorbing element, such as upon absorption of the amount of liquid removed from the pouch, the volume occupied by the absorbing element will change from a first absorbing element volume to a second absorbing element volume, such as wherein the second absorbing element volume is larger than the first absorbing element volume,
and wherein the pouch and the absorbing element are spatially arranged, such as located, relative to each other, such as initially spatially arranged relative to each other, so that there is an overlap between the first pouch volume and the second absorbing element volume, such as so that a volume initially occupied by the pouch can subsequently become occupied by the absorbing element via
removal of liquid from the pouch, such as emptying of the liquid initially placed in the pouch, and
absorption of liquid by the absorbing element.

According to an embodiment, there is presented a cartridge, wherein there is a free path along a rectilinear path from the pouch to the absorbing element. By having a free path along a rectilinear path (such as a line-of-sight) it may be possible for a liquid to move along such free path from the pouch to the absorbing element, which may in turn render it more likely that liquid leaked from the pouch can arrive, optionally even by freely flowing under the influence of gravity and/or capillary forces, at the absorbing element. It may be understood that the pouch and the absorbing element are not physically separated by solid material.

According to an embodiment, there is presented a cartridge, wherein the cartridge is arranged so that upon the absorbing element absorbing a liquid, the pouch and the absorbing element can be adjoining. An advantage of this may be that it enables exploiting the space in the cartridge optimally by not wasting space between the pouch and the absorbing element, at least upon the absorbing element absorbing a liquid.

According to an embodiment, there is presented a cartridge, wherein the pouch is adjoining the absorbing element during normal use.

According to an embodiment, there is presented a cartridge, wherein the pouch is adjoining the absorbing element. An advantage of this may be that it enables exploiting the space in the cartridge optimally by not wasting space between the pouch and the absorbing element. Another advantage may be that by the adjoining of the pouch and the absorbing element may increase a chance that liquid leaked from the pouch arrives at the absorbing element.

According to an embodiment, there is presented a cartridge, wherein the pouch is adjoining the absorbing element when the absorbing element is in a dry state. An advantage of this may be that this enables arranging the pouch and absorbing element as close to each other as possible (adjoining) and hence save space already at a stage where no liquid has been absorbed by the absorbing element.

According to an embodiment, there is presented a cartridge, wherein the absorbing element comprises a cellulosic products, such a cellulose acetate, a fiber-based products, a hydrocolloid, bentonite, sodium polyacrylate, a gelling agent, a thickening agent and/or a melamine resin foam. An advantage thereof might be that such absorbing element may be capable of fixating liquid, such as waste liquid (e.g., from an associated analysis unit) and/or liquid leaked from a pouch.

According to an embodiment, there is presented a cartridge, wherein the liquid comprised by the pouch is an aqueous reference liquid, an aqueous calibration liquid, an aqueous rinsing liquid, a control liquid, such as a quality control (QC) liquid, and/or an aqueous cleaning liquid. This may be advantageous, e.g., for enabling supplying the relevant liquid to an associated analysis unit. One or more or all of these aqueous liquids may be comprising constituents and/or parameters in the same or similar concentration levels (and also somewhat higher and lower, such as +/−1%, such as +/−10%) as the liquid being analyzed using the apparatus. Alternatively, or additionally, pH of one or more or all of these aqueous liquids may be within the range 5-9, such as 6-9.

According to an embodiment, there is presented a cartridge, wherein the pouch and the absorbing element are comprised within an interior volume of the cartridge, such as within an enclosure of the cartridge. This may be relevant for the purpose of absorbing liquid inside the cartridge, e.g., so as to avoid or reduce a risk of said liquid (being, e.g., waste and/or leaked liquid) leaving the cartridge.

According to an embodiment, there is presented a cartridge, wherein the absorbing element comprises a plurality of parts or portions some of which are comprised within a volume exterior to an interior volume of the cartridge. This may be relevant for the purpose of absorbing liquid spilled during connection of the cartridge to an associated analysis unit and/or during de-connection of the cartridge from an associated analysis unit.

According to an embodiment, there is presented a cartridge, wherein the cartridge comprises a lid. This may be advantageous for enabling closing the cartridge with the lid and providing a degree of protection of, e.g., the pouch (and optionally any liquid in the pouch) and the absorbing element and/or of personnel outside of the pouch, e.g., with respect to potentially biohazardous (waste) liquid in the cartridge.

According to an embodiment, there is presented a cartridge, wherein the liquid outlet port (of the cartridge) is fluidically connected to the pouch via one or more needles arranged for penetrating from a volume exterior to the pouch to an interior volume of the pouch. This may be advantageous for facilitating a fluidical connection from the inside of the pouch to the exterior of the pouch, such as doing so in a simple and/or controlled manner.

According to an embodiment, there is presented a cartridge, wherein the needles are attached to the lid and arranged for penetrating from a volume exterior to the pouch to an interior volume of the pouch upon closing of the lid. This may be advantageous for ensuring that the needles are readily available at or within the cartridge.

According to an embodiment, there is presented a cartridge, wherein the pouch comprises a multi-layered laminate. This may be relevant for, e.g., maintaining a certain partial pressure of a gas in the liquid. For example, the interior layer of the pouch contacting the liquid can be polyethylene and the exterior can be aluminum foil. This combination has proven effective in maintaining the partial pressure of oxygen gas in solution for a period of storage and use.

According to an embodiment, the multi-layered laminate comprises one or more metallic layers, such as aluminum and/or AlOx, and one or more polymeric layers on one side of the one or more metallic layers (such as the inside or the outside) or one or more polymeric layers on each side of the one or more metallic layers. An outer polymer layer may be advantageous for forming a shield against the exterior of the pouch, such as during manufacturing, storing, shipment and use. An inner polymer layer may be advantageous for enabling closing the pouch by melting at least a part of the inner polymeric layer. An inner polymer layer may furthermore be advantageous for increasing a gas tightness of the pouch and/or reducing a water vapor transmission rate. The polymeric material may comprise or consist of polyethylene, such as a polyethylene comprising both high density polyethylene (possibly being beneficial for improving gas protection layer properties) and low-density polyethylene (possibly being beneficial for improving weldability).

According to an embodiment, there is presented a cartridge, wherein the pouch, such as the at least one pouch, is gas-tight. A possible advantage may be that the liquid in the at least one pouch remains stable, such as enables use even after an extended period of time during storage and/or use.

By ‘gas tight’ may be understood, according to some embodiments, that a diffusion of oxygen through the pouch, such as through the pouch sidewall, is less than 5 cm³/m²/24 hrs/atm (where ‘hrs’ is hours and ‘atm’ is ‘atmosphere’, i.e., 101325 Pa), such as less than 3 cm³/m²/24 hrs/atm, such as less than 1 cm³/m²/24 hrs/atm, such as less than 0.5 cm³/m²/24 hrs/atm, such as less than 0.3 cm³/m²/24 hrs/atm, such as less than 0.1 cm³/m²/24 hrs/atm, such as less than 0.01 cm³/m²/24 hrs/atm, such as less than 0.001 cm³/m²/24 hrs/atm, as determined at room temperature 23 degree Celsius according to a Standard Test Method for Oxygen Gas Transmission Rate Through Plastic Film and Sheeting Using a Coulometric Sensor, such as according to ASTM D3985 in any version thereof, and in particular according to ASTM D3985-95 or alternatively according to ASTM D3985—05(2010)e.

Where ‘WVTR’ is Water Vapor Transmission Rate and ‘ASTM’ is American Society for Testing and Materials.

By ‘gas tight’ may alternatively or additionally be understood that WVTR is equal to or less than 5.0 g/m²/24 hrs, such as equal to or less than 1.0 g/m²/24 hrs, such as equal to or less than 0.5 g/m²/24 hrs, such as equal to or less than 0.1 g/m²/24 hrs, as determined at 38 degree Celsius and 90% Relative Humidity (RH) and/or according to ASTM 9932:1990.

An example of a pouch material may be given by aluminum foil having barrier properties given by:
WVTR: <0.1 g/m²/24 hrs (38 degree Celsius, 90% RH) ASTM 9932:1990
O₂: <0.1 cm³/m²/24 hrs/atm (23 degree Celsius, Carrier-humid, O₂ dry) ASTM 3985-95

Another example of a pouch material may be given by an aluminum Oxide (AlOx) foil having barrier properties given by:
WVTR: <0.1 g/m²/24 hrs (38 degree Celsius, 90% RH) ASTM 9932:1990
O₂: <0.3 cm³/m²/24 hrs/atm (23 degree Celsius, Carrier-humid, Oz dry) ASTM D 3985-95

According to an embodiment, there is presented a cartridge, wherein the pouch comprises a volume of liquid being
1 mL or more, such as 10 mL or more, such as 100 ml or more, and/or
5000 mL or less, such as 1000 mL or less, such as 500 mL or less, such as 200 ml or less.

An advantage of this may be that the pouch comprises an appropriate amount of liquid, such as for purposes related to fluid analysis, such as in a bio-medical or clinical setting.

According to an embodiment, there is presented a cartridge, wherein the absorbing element is capable of absorbing a volume of water being
1 mL or more, such as 10 mL or more, such as 100 mL or more, such as 1000 ml or more, and/or
10000 mL or less, such as 8000 mL or less, such as 7000 mL or less, such as 5000 mL or less, such as 2000 mL or less, such as 1000 ml or less.

An advantage of this may be that the absorbing element is capable of absorbing an appropriate amount of water, such as for purposes related to fluid analysis, such as in a bio-medical or clinical setting.

According to an embodiment, there is presented a cartridge, wherein the absorbing element is capable of absorbing a volume of water corresponding to more than 20%, such as 25% or more, such a 50% or more, such as 75% or more, such as 90% or more, of the total liquid volume within the pouch, such as the one or more pouches. An advantage of this may be that a substantial portion of the liquid from a leakage from the one or more pouches can be absorbed by the absorbing element.

According to an embodiment, there is presented a cartridge, wherein the absorbing element is capable of absorbing a volume of water corresponding to at least the total liquid volume within the pouch, such as the one or more pouches. An advantage of this may be that even the liquid from a total leakage of all liquid(s), such as all liquid(s) from the one or more pouches, can be absorbed by the absorbing element.

According to an embodiment, there is presented a cartridge, wherein the absorbing element is capable of absorbing a volume of water corresponding to more than the total liquid volume within the pouch, such as the one or more pouches, such as is capable of absorbing a volume of water corresponding to at least 110%, such as at least 125%, such as at least 150%, such as at least 200%, such as at least 500%, such as at least 1000%, of said total liquid volume. A possible advantage is that such overcapacity may introduce a safety buffer absorption capacity and/or (further) reduce a risk of (excess) liquid leaving the cartridge.

According to an embodiment, there is presented a cartridge, wherein the cartridge is suitable for being connected to an apparatus for analyzing one or more fluids, such as for determining one or more blood gas parameters, such as one or more of pH and pCO₂ and pO₂, and for providing the liquid initially placed in the pouch from the pouch to the apparatus via the liquid outlet port (of the cartridge) and for receiving a waste liquid from the apparatus to the cartridge via the liquid inlet port (of the cartridge).

According to an embodiment of an apparatus according to the first aspect, there is presented an apparatus, wherein the cartridge is a cartridge according to the second aspect.

According to a third aspect, there is presented a method for providing a liquid to an analysis unit, such as the analysis unit according to first aspect (such as to the analysis unit of—and as described in the context of—the apparatus according to the first aspect and/or the seventh aspect), for analyzing one or more fluids, said method comprising:
Providing the analysis unit, such as the analysis unit according to the first aspect,
Providing a cartridge, such as the cartridge according to the second aspect, comprising:
   i. A pouch, such as one or more pouches, such as two or more pouches, such as three or more pouches, such as 5 or more pouches, each comprising a liquid, such as an aqueous liquid,
   ii. An absorbing element, wherein the absorbing element is capable of absorbing water, such as capable of absorbing liquid water and/or liquid blood, and wherein the absorbing element is arranged exterior to the pouch and fluidically connected to an exterior surface of the pouch,
   iii. A liquid outlet (of the cartridge) port being fluidically connected to the pouch, and
   iv. A liquid inlet port (of the cartridge) being fluidically connected to the element,
Connecting, such as reversibly connecting, the cartridge to the analysis unit,
Providing the liquid initially placed in the pouch from the pouch to the analysis unit via the liquid outlet port, and
Optionally receiving a waste liquid from the analysis unit to the cartridge via the liquid inlet port (of the cartridge).

According to an embodiment, there is presented a method further comprising
receiving a waste liquid from the analysis unit to the cartridge via the liquid inlet port, and
absorbing the waste liquid with the absorbing element, such as wherein the absorbing element is being arranged external to each pouch of the cartridge and/or wherein the absorbing element is not being encapsulated by the pouch.

According to an embodiment, there is presented a method further comprising analyzing the fluid with the analysis unit.

According to a fourth aspect, there is presented use of a cartridge according to the second aspect, for providing a liquid to an analysis unit, such as the analysis unit for analyzing one or more fluids according to the first aspect, and optionally for analyzing a fluid.

According to a fifth aspect, there is presented use of an apparatus according to the first aspect of the invention for analysis of a fluid, such as a fluid comprising or consisting of blood, such as animal, such as mammalian, or human blood, such as a human whole blood sample.

In the context of point-of-care measurement systems (in the art also referred to as ‘bedsite’ systems) and laboratory environments alike, analysis, such as blood gas analysis, is oftentimes undertaken by users, such as nurses, who may not be users trained in use of, e.g., blood gas analyzers.

According to a sixth aspect of the invention (or an embodiment of the fourth aspect), there is presented use of a use of an apparatus according to the first aspect of the invention for point-of-care (POC) analysis, such as determination of one or more concentrations of one or more analytes, of a sample, such as a sample being a liquid whole blood sample.

POC measurement is also referred to as ‘bed site’ measurement in the art. In the present context, the term ‘point-of-care measurement’ should be understood to mean measurements which are carried out in close proximity to a patient, i.e., measurements that are not carried out in a laboratory. Thus, according to this embodiment, the user of the apparatus, such as the apparatus being a blood gas analyzer, performs measurement of a whole blood sample in a handheld blood sample container in the proximity of the patient, from whom the blood sample is taken, e.g., in the hospital room or ward accommodating the patient's bed, or in a nearby room of the same hospital department. In such use, the level of expertise of the user oftentimes varies from novice to experienced, and the capability of the blood gas analyzer to automatically output instructions matching each individual user's skills on the basis of sensor input is thus particularly beneficial in such environments.

According to a seventh second aspect, there is provided an apparatus for analysing one or more fluids, the apparatus comprising:
An analysis unit for analyzing one or more fluids, such as a body fluid, such as blood, such a mammalian blood, such as human blood, the analysis unit comprising:
   i. A liquid inlet port, and
   ii. A liquid outlet port, and
a cartridge according to the second aspect, with
   i. The liquid outlet port, such as the liquid outlet port of the cartridge, being fluidically connected to the pouch, and
   ii. The liquid inlet port, such as the liquid inlet port of the cartridge, being fluidically connected to the absorbing element,
wherein
the liquid inlet port of the analysis unit is fluidically connected with the liquid outlet port of the cartridge, and
the liquid outlet port of the analysis unit is fluidically connected with the liquid inlet port of the cartridge.

It may be understood that according to this embodiment, liquid exiting the analysis unit via the liquid outlet port of the analysis unit may be absorbed by the absorbing element in the cartridge, such as wherein the absorbing element is being arranged external to each pouch of the cartridge and/or wherein the absorbing element is not being encapsulated by the pouch.

According to an embodiment, there is presented an apparatus, wherein (the apparatus is arranged so that, such as arranged so that during use) liquid entering the analysis unit through the liquid inlet port of the analysis unit is fluidically decoupled or separated from liquid leaving the analysis unit through the liquid outlet port of the analysis unit. An advantage of this may be that liquid entering the analysis unit through the inlet port of the analysis unit is not mixed, such as contaminated and/or diluted, with liquid leaving the analysis unit through the liquid outlet port of the analysis unit.

According to an embodiment, the pouch, such each of the one or more pouches, is fluidically connected in a fluid-tight manner through the liquid outlet port of the cartridge to the liquid inlet port of the analysis unit and the liquid outlet port of the analysis unit is fluidically connected in a fluid-tight manner with the liquid inlet port of the cartridge, which is fluidically connected with the absorbing element.

According to an embodiment, there is presented an apparatus, wherein
the liquid inlet port of the analysis unit is fluidically connected with the liquid outlet port of the cartridge so that the analysis unit can receive, such as is arranged for receiving, liquid initially comprised within the pouch, and/or
the liquid outlet port of the analysis unit is fluidically connected with the liquid inlet port of the cartridge so that the analysis unit can output liquid which can be absorbed by the absorbing element, such as wherein the apparatus is arranged so that some or all liquid output from the analysis unit will become absorbed by the absorbing element.

According to an eighth aspect, there is presented a method for providing a liquid to the analysis unit according to any of the first aspect and/or the seventh aspect (such as to the analysis unit of—and as described in the context of—the apparatus according to the first aspect and/or the seventh aspect), said method comprising:
Providing the analysis unit according to the first aspect and/or the seventh aspect, such as the analysis unit of the apparatus according to the first aspect and/or the seventh aspect,
Providing the cartridge according to the second aspect,
Connecting, such as reversibly connecting, the cartridge to the analysis unit,
Providing the liquid initially placed in the pouch from the pouch to the analysis unit via the liquid outlet port, such as via the outlet port of the cartridge, and
optionally receiving a waste liquid from the analysis unit to the cartridge via the liquid inlet port, such as via the liquid inlet port of the cartridge
optionally absorbing the waste liquid with the absorbing element.

The first, second, third, fourth, fifth and sixth, seventh and eighth aspect of the present invention may each be combined with any of the other aspects. These and other aspects of the invention will be apparent from and elucidated with reference to the embodiments described hereinafter.

### BRIEF DESCRIPTION OF THE FIGURES

The apparatus, cartridge, method and use according to the invention will now be described in more detail with regard to the accompanying figures. The figures show one way of implementing the present invention and is not to be construed as being limiting to other possible embodiments falling within the scope of the attached claim set.

Preferred embodiments of the invention will be described in more detail in connection with the appended drawings, which show in

FIG. 1 is a perspective view of a flexible pouch, which comprises a liquid and a gas tight material.

FIG. 2A is a perspective view of a cartridge comprising the pouch.

FIG. 2B is a cut-away view of the cartridge of FIG. 2A along line 2B-2B.

FIG. 3 shows the cartridge of FIG. 2 secured to an analysis unit, such as a blood analysis apparatus.

FIG. 4 shows a perspective view of a part of a cartridge comprising two pouches.

FIG. 5 is a cut-away view similar to FIG. 2B of an embodiment of a cartridge.

### DETAILED DISCLOSURE OF THE INVENTION

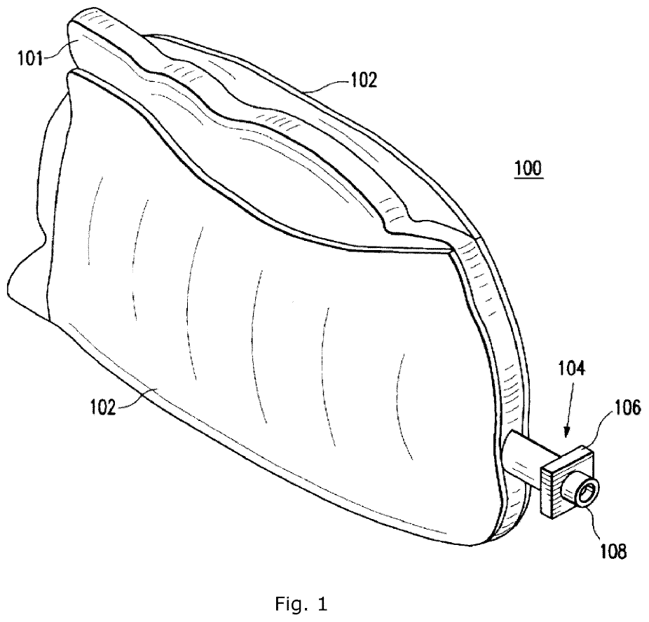

FIG. 1 is a perspective view of a pouch 100. The pouch 100 holds a reference solution for use in calibrating an analysis unit, such as an analysis unit for analyzing blood. The pouch 100 has a multi-layered laminate 101 and a self-sealing liquid port 104 for enabling the analysis unit to repeatedly receive reference solution from the pouch 100. The multi-layered laminate 101 is a flexible, metal-plastic laminate. The liquid port 104 (which is described in detail in the application U.S. Pat. No. 5,777,202, which is hereby incorporated by reference in entirety) has a flange 106, a port body 108 for engaging and fluidically connecting to a liquid outlet port of a cartridge, and a fluid communication port (i.e., the through-going opening in the port body 108. The pouch 100 may optionally also have a protective layer 102 covering a substantial portion of the exterior of the pouch 100.

The multi-layered laminate 101 is made from a flexible material, preferably an aluminum oxide (AlOx) foil-plastic laminate or an aluminum foil-plastic laminate.

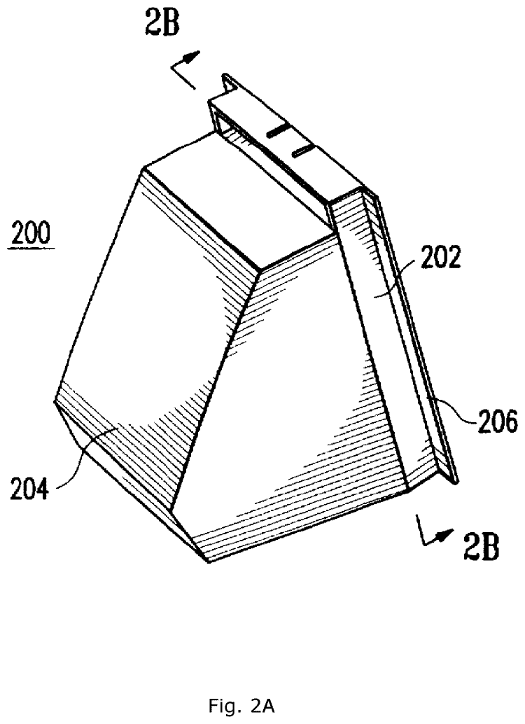

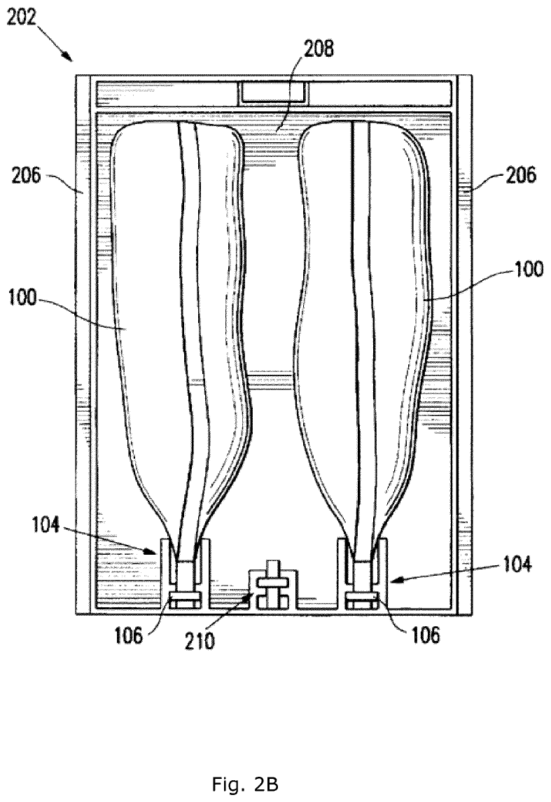

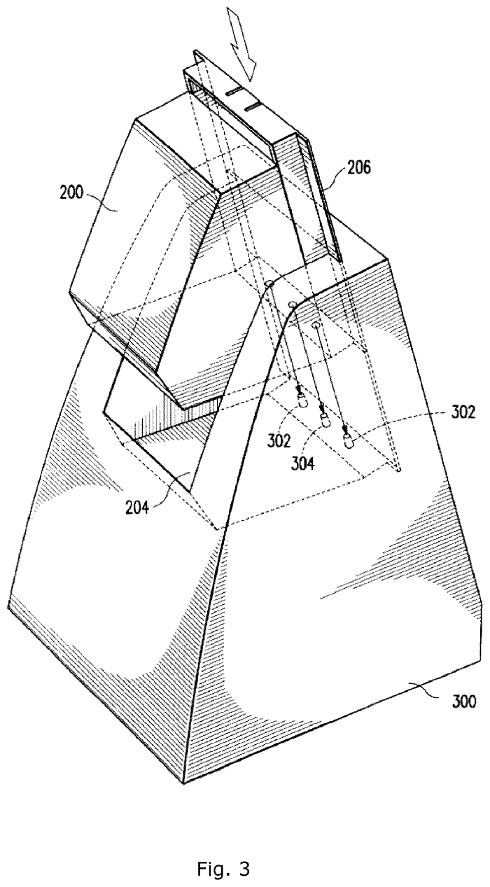

FIG. 2A shows a cartridge 200 that can be employed to hold one or more pouches 100. The cartridge 200 may have a tray 202 and cover 204 that are secured together to enclose the pouches 100. The cartridge illustrated in FIG. 2 is merely exemplary. It should be recognized that the cartridge 200 may have almost any shape and need not completely enclose the pouches 100, yet in embodiments it does completely enclose the pouch or pouches. The cartridge may have at least one rail 206 for aligning the cartridge 200 with a housing for an analysis unit, such as a blood analysis device, as will be described with reference to FIG. 3.

The cartridge 200 may be made from plastic or any other suitable material. A more detailed description of an exemplary cartridge can be found in a U.S. patent application Ser. No. 08/650,340, filed May 20, 1996, entitled “Integral Fluid and Waste Container for Blood Analyzer”, which is hereby included by reference in entirety.

FIG. 2B is a cut-away view of the cartridge 200 along line 2B-2B of FIG. 2A. This view shows the tray 202 holding two pouches 100 and an absorbing element 208. The absorbing element in the present embodiment is a sheet material placed at boundary wall of the tray 202 (and hence of the cartridge 200) between the pouches and the boundary wall (such as on the side of the boundary wall facing the interior of the cartridge). The absorbing element is adjoining the pouches (at a side of the pouches facing the boundary wall). The absorbing element 208 collects waste liquids (such as blood and/or used reference solution) from the analysis unit, such as a blood analysis device. The pouches 100, being preferably flexible, contract as reference solution exits the pouches 100. As the pouches 100 contract, they leave space in the cartridge 200, which may be filled by the absorbing element as it expands with waste liquid, e.g., due to the absorbing element absorbing waste liquid and converting the incoming waste liquid into a substantially solid material.

FIG. 3 shows the housing of an analysis unit, such as a blood analyzer, 300 with the cartridge 200 inserted into a cavity in the analysis unit 300. The rails help guide the cartridge 200 into the analysis unit 300, such that the liquid port 104 for the reference solution bag 100 and the liquid inlet port 210 of the cartridge for the absorbing element 208 will properly engage fluid carrying devices or fittings, such as liquid inlet port 302 of the analysis unit 300 and liquid outlet port 304 of the analysis unit 300. Liquid inlet port 302 of the analysis unit is positioned on the analysis unit 300 to engage the liquid port 104 on the reference solution bag 100 (so reference solution can pass from the pouch 100 via a liquid outlet port of—and being an opening in—the cartridge), and liquid outlet port (fluid fitting) 304 of the analysis unit is positioned to engage with a liquid inlet port 210 of the cartridge 200 and thus enabling providing waste liquid from the analysis unit 300 to the absorbing element 208. The liquid inlet port and outlet port (fluid fittings) 302, 304 of the analysis unit protrude from and are part of the analysis unit 300. As such, the liquid inlet port and outlet port 302, 304 of the analysis unit constitute fluid carrying devices that are external to the pouches 100 and the absorbing element 208. A more detailed description of an exemplary analysis unit in the form of a blood analyzer can be found in a U.S. patent application Ser. No. 08/650,341, entitled “Portable Modular Blood Analyzer with Simplified Fluid Handling Sequence,” filed May 20, 1996, which is hereby included by reference in entirety.

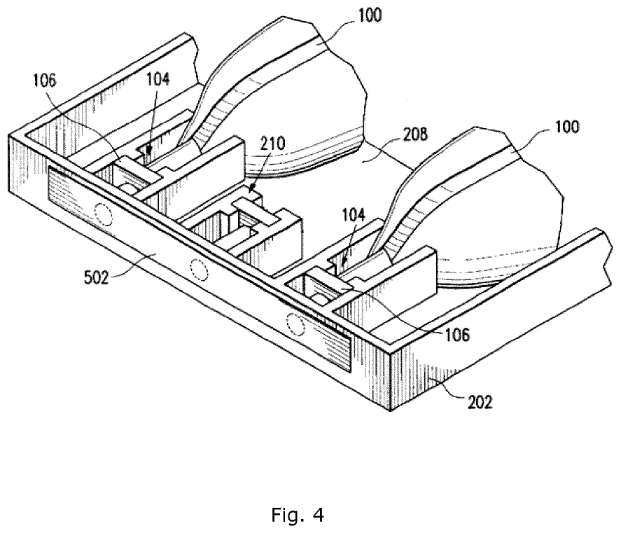

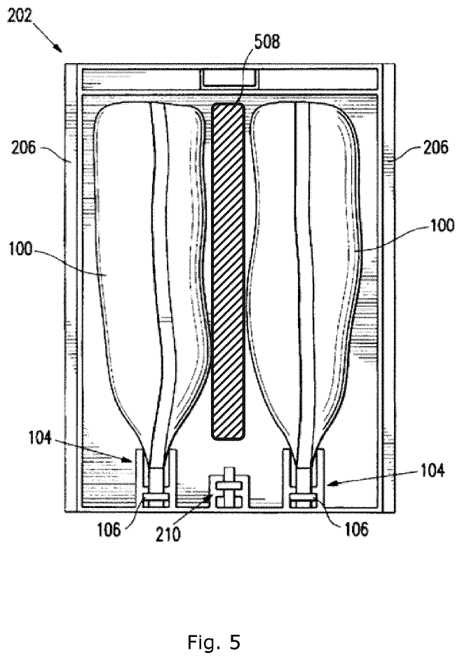

FIG. 4 shows tray 202 with foil tape 502 covering the self-sealing liquid port (or valves) 104 and the associated liquid outlet port of the cartridge (being the opening in the cartridge allowing fluidically connecting liquid port 104 on the pouch with liquid inlet port 302 on the analysis unit) and waste liquid inlet port 210 of the cartridge. It should be understood, however, that the foil tape 502 need not be a single piece, like that shown in FIG. 5, but instead could be independent pieces covering each port from which it is desirable to protect against the escape of gases and to prevent dirt from interfering with the liquid port (valves) 104 and liquid inlet port 210 of the cartridge. When the cartridge 200 is ready to be inserted into the analysis unit 300, the foil tape 502 is preferably first removed, although the cartridge 200 can be inserted into the analysis unit 300 with the foil 502 in place, and the liquid inlet- and outlet ports (fittings) 302, 304 would then puncture the foil tape 502 when inserted into the liquid port (valves) 104 and liquid inlet port 210 of the cartridge. Those skilled in the art will recognize that the foil tape 502 is merely an exemplary means by which the self-sealing liquid port (valves) 104 and liquid inlet port 210 of the cartridge can be further sealed against the escape or ingress of gases. Other means include metal or plastic plugs or caps.

With the foil tape 502 in place, the pouches 100 are even further substantially sealed against the communication of gases through the liquid port (valve) 104.

The pouches 100, when full of liquid, such as reference solution, and the empty absorbing element 208, preferably fit relatively snugly within the cartridge 200. Thus, during transport and storage (e.g., when the cartridge 200 has not been used), the pouches 100 are constrained by the walls of the cartridge 200. As the pouches 100 are emptied during, e.g., blood tests, the space they leave within the cartridge 200 from their decreasing internal volume is filled by the expanding absorbing element 208, which holds, e.g., the waste blood and waste reference solution from the analysis unit, such as the blood analyzer, 300. Accordingly, the pouches 100 remain constrained, now by the walls of the cartridge 200 and the expanding absorbing element 208, as the pouches 100 are emptied of liquid.

FIG. 5 shows a tray 202, which is similar to the tray depicted in FIG. 2 except that the absorbing element (sheet) 208 of FIG. 2 has been replaced in the embodiment of FIG. 5 with an absorbing element extending into a space between the pouches 100. The absorbing element is adjoining the pouches (at a side of the pouches facing each other). In an alternative embodiment, the absorbing element and pouches are initially not adjoining each other, but optionally being arranged for adjoining upon absorption of liquid by the absorbing element.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

According to items 1-15 below, there is presented embodiments, such as alternative embodiments, which are combinable with any of the subject-matter, such as features and/or embodiments, in the remainder of the description and/or in the appended claims:
1. An apparatus for analyzing one or more fluids, the apparatus comprising:
   An analysis unit for analyzing one or more fluids, such as a body fluid, such as blood, such a mammalian blood, such as human blood, the analysis unit comprising:
      i. A liquid inlet port, and
      ii. A liquid outlet port, and
   a cartridge comprising:
      i. A pouch, such as one or more pouches, such as two or more pouches, such as three or more pouches, such as 5 or more pouches, each comprising a liquid, such as an aqueous liquid,
      ii. An absorbing element, wherein the absorbing element is capable of absorbing water, such as capable of absorbing liquid water and/or liquid blood, and wherein the absorbing element is arranged exterior to the pouch and fluidically connected to an exterior surface of the pouch,
      iii. A liquid outlet port being fluidically connected to the pouch, and
      iv. A liquid inlet port being fluidically connected to the absorbing element,
   wherein
   the liquid inlet port of the analysis unit is fluidically connected with the liquid outlet port of the cartridge, and
   the liquid outlet port of the analysis unit is fluidically connected with the liquid inlet port of the cartridge.
2. The apparatus according to item 1, wherein the one or more fluids comprise or is human blood.
3. The apparatus according to any of the preceding items, wherein the analysis unit is suitable for determining one or more blood gas parameters, such as one or more of pH, pO₂, pCO₂.
4. The apparatus according to according to any of the preceding items, wherein the analysis unit comprises an ion selective electrode membrane sensor, such as an ion selective electrode membrane sensor adapted for sensing one or more blood gas parameters.
5. A cartridge comprising:
   A pouch, such as one or more pouches, such as two or more pouches, such as three or more pouches, such as 5 or more pouches, each comprising a liquid, such as an aqueous liquid,
   An absorbing element being capable of absorbing water, such as capable of absorbing liquid water and/or liquid blood, and arranged exterior to the pouch and fluidically connected to an exterior surface of the pouch,
   A liquid outlet port being fluidically connectable to the pouch and/or fluidically connected to the pouch,
   A liquid inlet port being fluidically connectable to the absorbing element and/or fluidically connected to the element.
6. The cartridge according to item 5, wherein the pouch and absorbing element are arranged so that in case liquid initially comprised within the pouch is leaked to the exterior of the pouch, said liquid initially comprised within the pouch is able to be absorbed by the absorbing element subsequent to leaking to the exterior of the pouch, such as at least in one orientation of the cartridge.
7. The cartridge according to any of items 5-6, wherein the pouch and the absorbing element are arranged so that a volume initially occupied by the pouch can subsequently become occupied by the absorbing element via
   removal of liquid from the pouch, such as emptying of the liquid initially placed in the pouch, and
   absorption of liquid by the absorbing element.
8. The cartridge according to any of items 5-7, wherein the pouch is adjoining the absorbing element.
9. The cartridge according to any of items 5-8, wherein the absorbing element comprises a cellulosic product, such a cellulose acetate, a fiber-based product, a hydrocolloid, bentonite, sodium polyacrylate, a gelling agent, a thickening agent and/or a melamine resin foam.
10. The cartridge according to any of items 5-9, wherein the liquid comprised by the pouch is an aqueous reference liquid, an aqueous calibration liquid, an aqueous rinsing liquid, and/or an aqueous cleaning liquid.
11. The cartridge according to any of items 5-10, wherein the liquid outlet port is fluidically connected to the pouch via one or more needles arranged for penetrating from a volume exterior to the pouch to an interior volume of the pouch.
12. The cartridge according to any of items 5-11, wherein the pouch comprises a multi-layered laminate and/or wherein the pouch is gas-tight.
13. The apparatus according to any of items 1-4, wherein the cartridge is the cartridge according to any of items 5-12.
14. A method for providing a liquid to an analysis unit, such as the analysis unit according to any of items 1-4, for analyzing one or more fluids, said method comprising:
   Providing the analysis unit, such as the analysis unit according to any of items 1-4,
   Providing a cartridge, such as the cartridge according to any of items 5-12, comprising:
      i. A pouch, such as one or more pouches, such as two or more pouches, such as three or more pouches, such as 5 or more pouches, each comprising a liquid, such as an aqueous liquid,
      ii. An absorbing element, wherein the absorbing element is being capable of absorbing water, such as capable of absorbing liquid water and/or liquid blood, and wherein the absorbing element is arranged exterior to the pouch and fluidically connected to an exterior surface of the pouch,
      iii. A liquid outlet port being fluidically connected to the pouch, and
      iv. A liquid inlet port being fluidically connected to the element,
   Connecting, such as reversibly connecting, the cartridge to the analysis unit,
   Providing the liquid initially placed in the pouch from the pouch to the analysis unit via the liquid outlet port, and
   optionally receiving a waste liquid from the analysis unit to the cartridge via the liquid inlet port.
15. Use of a cartridge according to any of items 5-12 for providing a liquid to an analysis unit, such as the analysis unit for analyzing one or more fluids according to any of items 1-4, and optionally for analyzing a fluid.

1. A cartridge comprising:
(a) one or more pouches, each comprising a liquid,
(b) an absorbing element capable of absorbing liquid arranged exterior to at least one pouch of the one or more pouches and fluidically connected to an exterior surface of at least one pouch of the one or more pouches,
(c) a liquid outlet port being either fluidically connectable to at least one pouch of the one or more pouches or fluidically connected to at least one pouch of the one or more pouches, and
(d) a liquid inlet port being either fluidically connectable to the absorbing element or fluidically connected to the absorbing element.

2. The cartridge according to claim 1, wherein the absorbing element is arranged external to each pouch of the one or more pouches.

3.-4. (canceled)

5. The cartridge according to claim 1, wherein the absorbing element is capable of absorbing a volume of water corresponding to at least a total liquid volume within at least one pouch of the one or more pouches.

6. The cartridge according to claim 1, the one or more pouches comprising two or more pouches, each pouch of the two or more pouches comprising a liquid, the liquid outlet port being either fluidically connectable to each pouch of the two or more pouches or fluidically connected to each pouch of the two or more pouches.

7. The cartridge according to claim 1, the one or more pouches comprising four or more pouches, each pouch of the four or more pouches comprising a liquid, the liquid outlet port being either fluidically connectable to each pouch of the four or more pouches or fluidically connected to each pouch of the four or more pouches.

8. (canceled)

9. The cartridge according to claim 1, wherein the cartridge has a size along its largest outer dimension of 25 cm or less.

10. The cartridge according to claim 1, wherein the cartridge has a size along its largest outer dimension of 5 cm or more.

11. The cartridge according to claim 1, wherein the cartridge comprises an enclosure, wherein the enclosure encloses at least the one or more pouches and the absorbing element, wherein either or both of the liquid outlet port or the liquid inlet port is accessible from outside of the enclosure.

12. The cartridge according to claim 11, wherein the enclosure is more rigid than each pouch of the one or more pouches.

13. (canceled)

14. The cartridge according to claim 1, wherein the liquid outlet port is fluidically connected with the liquid inlet port.

15. The cartridge according to claim 1, wherein the liquid outlet port is fluidically separated with respect to the liquid inlet port.

16. The cartridge according to claim 1, wherein each pouch of the one or more pouches and the absorbing element are arranged so that in case liquid initially comprised within at least one pouch of the one or more pouches is leaked to the exterior of the at least one pouch, said liquid initially comprised within the at least one pouch is able to be absorbed by the absorbing element subsequent to leaking to the exterior of the at least one pouch, in at least one orientation of the cartridge.

17. (canceled)

18. The cartridge according to claim 1, wherein at least one pouch of the one or more pouches and the absorbing element are arranged so that a volume initially occupied by the at least one pouch can subsequently become occupied by the absorbing element via:
removal of liquid from the at least one pouch, and
absorption of liquid by the absorbing element.

19. The cartridge according to claim 1, wherein
at least one pouch of the one or more pouches is arranged so that upon removal of liquid from the at least one pouch, the volume occupied by the p at least one ouch will change from a first pouch volume to a second pouch volume, wherein the second pouch volume is smaller than the first pouch volume, and
the absorbing element is arranged so that upon absorption of liquid by the absorbing element, the volume occupied by the absorbing element will change from a first absorbing element volume to a second absorbing element volume, wherein the second absorbing element volume is larger than the first absorbing element volume,
and wherein the at least one pouch and the absorbing element are spatially arranged relative to each other so that there is an overlap between the first pouch volume and the second absorbing element volume, such as so that a volume initially occupied by the at least one pouch can subsequently become occupied by the absorbing element via:
removal of liquid from the at least one pouch, and
absorption of liquid by the absorbing element.

20. The cartridge according to claim 1, wherein at least one pouch of the one or more pouches is adjoining the absorbing element.

21. (canceled)

22. The cartridge according to claim 1, wherein the absorbing element comprises one or more materials selected from the group consisting of: cellulosic material, a fiber-based material, a hydrocolloid, bentonite, sodium polyacrylate, a gelling agent, a thickening agent, and a melamine resin foam.

23. The cartridge according to claim 1, wherein at least one pouch of the one or more pouches contains one or more liquids selected from the group consisting of: an aqueous reference liquid, an aqueous calibration liquid, an aqueous rinsing liquid, and an aqueous cleaning liquid.

24. The cartridge according to claim 1, wherein the liquid outlet port is fluidically connected to at least one pouch of the one or more pouches via one or more needles arranged for penetrating from a volume exterior to the at least one pouch to an interior volume of the at least one pouch.

25. (canceled)

26. An apparatus for analyzing one or more fluids, the apparatus comprising:
(a) an analysis unit for analyzing one or more fluids, the analysis unit comprising:
(i) a liquid inlet port, and
(ii) a liquid outlet port, and
(b) a cartridge according to claim 1, with:
the liquid outlet port of the cartridge being fluidically connected to at least one pouch of the one or more pouches, and
the liquid inlet port of the cartridge being fluidically connected to the absorbing element,
wherein
the liquid inlet port of the analysis unit is fluidically connected with the liquid outlet port of the cartridge, and
the liquid outlet port of the analysis unit is fluidically connected with the liquid inlet port of the cartridge.

27.-29. (canceled)

30. An apparatus for analyzing one or more fluids, the apparatus comprising:
(a) an analysis unit for analyzing one or more fluids, the analysis unit comprising:
(i) a liquid inlet port, and
(ii) a liquid outlet port, and
(b) a cartridge comprising:
(i) one or more pouches, each comprising a liquid,
(ii) an absorbing element, wherein the absorbing element is capable of absorbing liquid, and wherein the absorbing element is arranged exterior to the one or more pouches and fluidically connected to an exterior surface of the one or more pouches,
(iii) a liquid outlet port being fluidically connected to the one or more pouches, and
(iv) a liquid inlet port being fluidically connected to the absorbing element,
wherein
the liquid inlet port of the analysis unit is fluidically connected with the liquid outlet port of the cartridge, and
the liquid outlet port of the analysis unit is fluidically connected with the liquid inlet port of the cartridge.

31.-38. (canceled)
